(19) United States
(12) Patent Application Publication
(10) Pub. No.: US 2024/0123252 A1
(43) Pub. Date: Apr. 18, 2024

(54) LIGHTING FIXTURE WITH SAFE UV RADIATION FOR VITAMIN D PRODUCTION

(71) Applicant: HAVELLS INDIA LIMITED, New Delhi (IN)

(72) Inventors: Ashwani Singh, New Delhi (IN); Gaurav Trigunayak, New Delhi (IN); Pushpraj Gir, New Delhi (IN); Ameet Gupta, New Delhi (IN)

(73) Assignee: HAVELLS INDIA LIMITED, New Delhi (IN)

(21) Appl. No.: 18/274,661

(22) PCT Filed: Jan. 13, 2023
(86) PCT No.: PCT/IB2023/050306

§ 371 Date: Jul. 27, 2023

(30) Foreign Application Priority Data
Jan. 24, 2022 (IN) 202211003985

(51) Int. Cl. A61N 5/06

(52) U.S. Cl. CPC A61N 5/0613; A61N 2005/0642; A61N 2005/0651; A61N 2005/0661

(57) ABSTRACT

The present invention provides a lighting fixture comprising a combination of LEDs capable of radiating in the visible wavelengths or UV wavelengths and a reflector to obtain a particular beam angle of light/radiation, features/design of which provides safe levels of UV radiation over a period of time to promote vitamin D production upon contact with exposed skin surface. The lighting fixture of the present invention is particularly suitable for indoor areas such as office spaces, where people spend anywhere from 6-10 hours without exposure to sun, which is a major source of radiation for vitamin D production in the body. The lighting fixture of the present invention addresses the issue of chronic/endemic vitamin D deficiency in the Indian population and provides a cheap, effective and safe way to obtain daily vitamin D dosage and its health benefits.

### FIELD OF INVENTION

The present invention relates generally the field of lighting technology. In particular, the invention relates to lighting technology with safe UV radiation for promoting vitamin D production.

### BACKGROUND OF THE PRESENT INVENTION

Vitamin D deficiency is endemic within the general Indian population with as high as 70% of the population suffering from varying levels of vitamin D deficiency. Even though vitamin D can be produced by the body by exposure of some part of the skin to the sun, due to a variety of reasons, Indians do not tend to expose themselves to the sun. Consequently, subclinical vitamin D deficiency is common especially in urban populations.

This problem is particularly exacerbated within the urban working population who spend 6-9 hours of daytime within an office environment where there is no sunlight and typically the only source of light is blue/yellow LEDs or tube lighting. Such lighting sources provide no radiation which is effective in inducing vitamin D production in the body.

A correlation between low vitamin D levels and various health issues such as mood swings, muscle weakness, cardiovascular disease, depression, obesity, etc. have been established. Therefore, there is a clear need to enhance vitamin D levels in the general population, especially in the office going community who do not/cannot get adequate exposure to the sun.

While there are many known lighting devices for vitamin D production in the body, all such devices are used medical settings, or are to be used in highly regulated environment for very brief amounts of time as the radiation emitted by them are typically at hazardous levels.

There is a need in the art to develop lighting systems which can be safely used over an extended period of time to provide the benefits of UV radiation to induce vitamin D production by the human body without any constant medical or professional oversight.

### SUMMARY OF THE INVENTION

In an aspect of the present invention, there is provided a lighting fixture comprising: (a) one or more LEDs emitting light in the visible wavelength; (b) one or more LEDs emitting light in the UV spectrum having wavelength peak value of 303 nm; and (c) a reflector, wherein upon exposure to UV light, AUV is not more than 0.00104 W/m², NUV is not more than 0.00056 W/m², and EUVB irradiance is not more than 0.01004 W/m² at 1.8 m from the light source.

In another aspect of the present invention, there is provided a method of providing light in the UVB wavelength for promoting Vitamin D production on exposed skin, said method comprising: (i) obtaining at least a lighting fixture comprising: (a) one or more LEDs emitting light in the visible wavelength; (b) one or more LEDs emitting light in the UV spectrum having wavelength peak value of 303 nm; and (c) a reflector, wherein upon exposure to UV light, AUV is not more than 0.00104 W/m², NUV is not more than 0.00056 W/m², and EUVB irradiance is not more than 0.01004 W/m² at 1.8 m from the light source; and (ii) exposing skin to said lighting fixture, wherein said method promotes Vitamin D production.

In yet another aspect of the present invention, there is provided a lighting fixture comprising: (a) multiple LEDs emitting light in the visible wavelength; (b) 12 LEDs emitting light in the UVB spectrum having wavelength peak value of 303 nm; and (c) an aluminum reflector, wherein the beam angle of LEDs emitting light in the UVB spectrum is 30°, the radiant power of a LED emitting light in the UV spectrum is 3.3 mW, and the visible light brightness of the fixture is 500 lux measured at 1.8 m from the light source.

This summary is not intended to identify essential features of the claimed invention nor is it intended for use in determining or limiting the scope of the claimed subject matter.

### BRIEF DESCRIPTION OF THE ACCOMPANYING DRAWINGS

The following drawings form part of the present specification and are included to further illustrate aspects of the present invention. The invention may be better understood by reference to the drawings in combination with the detailed description of the specific embodiments presented herein.

FIG. 1a shows the spectral irradiance of module of output 14.4 mW at various distances, in accordance with an embodiment of the present invention.

FIG. 1b shows the spectral irradiance of module of output 39.6 mW at various distances, in accordance with an embodiment of the present invention

FIG. 1c shows the spectral irradiance of module of output 72 mW at various distances, in accordance with an embodiment of the present invention

FIG. 1d shows the spectral irradiance of module of output 84 mW at various distances, in accordance with an embodiment of the present invention

FIG. 1e shows the total irradiance of modules of output 14.4 mW, 39.6 mW, 72 mW, and 84 mW at various distances in accordance with an embodiment of the present invention.

FIG. 1f shows the EUV values of modules of output 14.4 mW, 39.6 mW, 72 mW, and 84 mW at various distances in accordance with an embodiment of the present invention

FIG. 2 shows the vitamin D level at different heights with or without reflector for 84 mW total radiant power, in accordance with an embodiment of the present disclosure.

FIG. 3 shows the vitamin D level at different heights with or without reflector for 39.6 mW total radiant power, in accordance with an embodiment of the present disclosure.

FIG. 4 shows the illustrative depiction of installation of the light fixture of the present invention, in accordance with an embodiment of the present disclosure.

### DETAILED DESCRIPTION OF THE INVENTION

Those skilled in the art will be aware that the invention described herein is subject to variations and modifications other than those specifically described. It is to be understood that the invention described herein includes all such variations and modifications. The invention also includes all such steps, features, and methods referred to or indicated in this specification, individually or collectively, and any and all combinations of any two or more of said steps or features.

For convenience, before further description of the present invention, certain terms employed in the specification, examples are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

As used in the specification and the claims, the singular forms “a”, “an”, and “the” include plural referents unless the context clearly dictates otherwise.

The present disclosure is not to be limited in scope by the specific embodiments described herein, which are intended for the purposes of exemplification only.

Functionally-equivalent processes and methods are clearly within the scope of the disclosure, as described herein.

The present invention provides a lighting fixture comprising: (a) one or more LEDs emitting light in the visible wavelength; (b) one or more LEDs emitting light in the UV spectrum having wavelength peak value of 303 nm; and (c) a reflector, wherein upon exposure to UV light, AUV is not more than 0.00104 W/m², NUV is not more than 0.00056 W/m², and EUVB irradiance is not more than 0.01004 W/m² at 1.8 m from the light source.

In an embodiment, the lighting fixture comprises 5-20 LEDs emitting light in the visible wavelength. In an embodiment, the radiant power of at least a first LED emitting light in the visible wavelength can be different from at least a second LED emitting light in the visible wavelength. In a preferred embodiment, the radiant power of each LED emitting light in the visible wavelength are the same. The visible light brightness of the lighting fixture is in the range of 450-600 lux measured at 1.8 m from the light source. In a preferred embodiment, the brightness is 500 lux.

The radiant power of at least an LED comprised in the lighting fixture emitting light in the UV spectrum is 3.3 mW. The total radiant power of the LEDs comprised in the lighting fixture emitting light in the UV spectrum is 39.6 mW. In a preferred embodiment, the lighting fixture comprises 12 LEDs emitting light in the UV spectrum.

In an embodiment, the reflector is made of aluminum or PCTF. In a preferred embodiment, the reflector is made of aluminum. The beam angle of one or more LEDs emitting light in the UV spectrum is 30±5°. In a preferred embodiment, the beam angle is 30°.

In an embodiment, the lighting fixture is circular in shape. In another embodiment, the lighting fixture is oval in shape. In yet another embodiment, the lighting fixture is polygonal in shape. The polygonal shape can in particular be triangular, square or rectangular. It is understood that lighting fixtures with more than 4 edges are also covered within the said definition. It is understood by a person skilled in the art that the arrangement of the LEDs in the lighting fixture is not limited to any particular arrangement and that any arrangement as per design and requirement can be achieved without deviating from the inventive concept of the present invention.

In a preferred embodiment, the lighting fixture of the present invention further has a diffuser covering the LEDs such that individual LEDs are not visible from outside the lighting fixture. The diffuser has low UVB absorption coefficient so as to allow efficient transmission (60-90%) of UV radiation from the LEDs in the lighting fixture to external environment.

In a most preferred embodiment of the present invention, there is provided a lighting fixture comprising: (a) multiple LEDs emitting light in the visible wavelength; (b) 12 LEDs emitting light in the UVB spectrum having wavelength peak value of 303 nm; and (c) an aluminum reflector, wherein the beam angle of LEDs emitting light in the UVB spectrum is 30°, the radiant power of a LED emitting light in the UV spectrum is 3.3 mW, and the visible light brightness of the fixture is 500 lux measured at 1.8 m from the light source. The AUV is not more than 0.00104 W/m², NUV is not more than 0.00056 W/m², and EUVB irradiance is not more than 0.01004 W/m² at 1.8 m from the light source.

The present invention also provides a method of providing light in the UVB wavelength for promoting Vitamin D production on exposed skin, said method comprising: (a) obtaining at least a lighting fixture as substantially described in the present disclosure; and (b) exposing skin to said lighting fixture, wherein said method promotes Vitamin D production. The said method results in production of Vitamin D equivalent to 390 IU when skin (type IV) exposure is 18% of total skin and exposure time is 6 hours.

The lighting fixture of the present invention is particularly suitable for office settings, where there is not enough sunlight and people spend 6-9 hours of daytime, thus resulting in vitamin D deficiency. The lighting fixture allows for a person be exposed to safe amounts of UVB radiation over a period of 6 hours so as to provide a sufficient daily amount of vitamin D production to a person.

The lighting fixture of the present invention is compliant with UV hazard standard of IEC-62471, which is a photobiological safety evaluation standard of LED lighting devices. The lighting fixture of the present invention conforms to actinic UV hazards (AUV) and near-UV hazard (NUV) limits of 0.001 and 10 W/m² respectively, which is indicative of the fact that the lighting fixtures do not pose any health risk.

The lighting fixture of the present invention provides total UEV (erythemal weighted UV) dose of 216.86 J/m² when used for 6 hours, which provides 390 IU of vitamin D.

According to the IEC62471 standard, stability evaluation of general lighting must be measured at an illuminance of 500 lux. As per the National Building Code of India 2005, the average ceiling height of houses, apartments, and offices are 2.75 m and that general lighting should be able to provide a brightness of 500 lux at 1.8 m. Therefore, the present invention is designed to conform to established standards and codes and validation of the invention is based on the said established standards and codes.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs.

4 different types of UVB LED light sources were selected and their optical characteristics based on distances and applied currents were measured and analyzed. 4 different modules, each having UVB output of 14.4 mW (12×1.2 mW), 39.6 mW (12×3.3 mW), 72 mW (12×6 mW), and 84 mW (12×7 mW) were tested. The specification of the UVB LEDs is as shown in Table 1 below.

| Radiant power | Peak wavelength | Maximum current | Forward voltage |
|---|---|---|---|
| (mW) | (nm) | (mA) | (mV) |
| 1.2 | 310 | 30 | 5.5 |
| 7 | 310 | 200 | 6 |
| 6 | 311 | 50 | 5.5 |
| 3.3 | 303 | 30 | 6 |

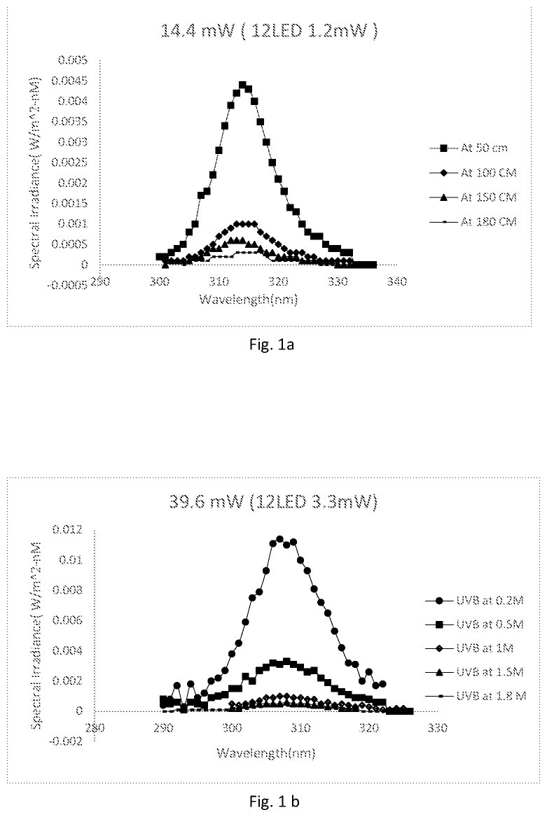

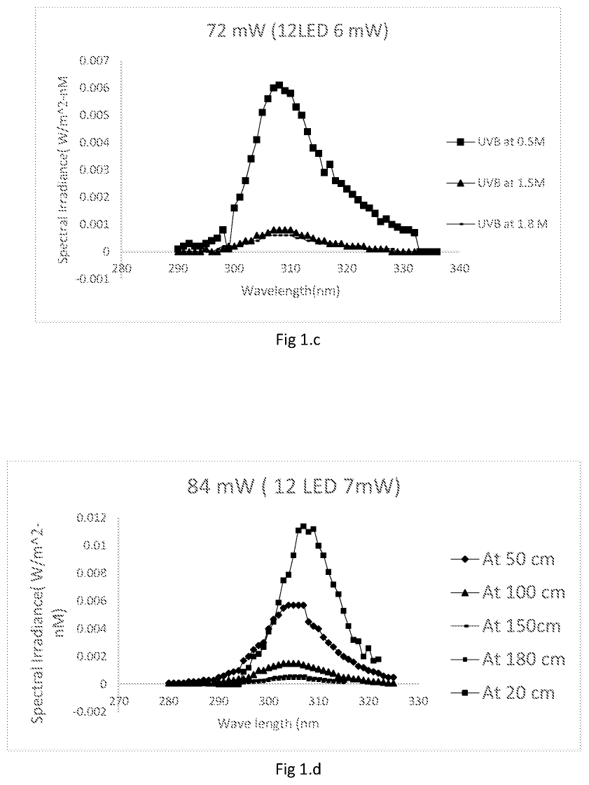

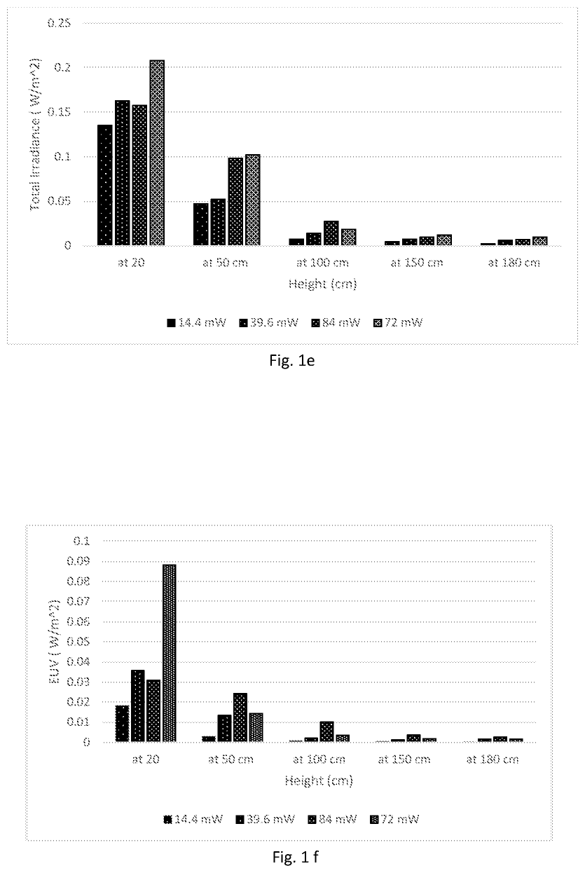

The light characteristics of the 4 modules were tested at distances of 20, 50, 100, 150, and 180 cm from the light source. FIG. 1a-d shows the results of the 14.4 mW UVB LED, 39.6 mW UVB LED, 72 mW UVB LED and 84 mW UVB LED modules. As expected, UVB irradiance decreases with distance. It is to be appreciated that at 1.8 m distance, which is the typical distance a person sits from a light source, the radiation risk is reduced.

FIG. 1e shows the total UVB irradiance (W/m²) of each of the 4 modules at various distances. FIG. 1f shows the EUV (UVB*weight function (IEC 62471). Weightage function is defined for every wavelength) (W/m²) of each of the 4 modules at various distances.

It has been shown previously that a EUV dose of 105 J/m² is required to meet 200 IU of vitamin D when 6-10% of the body is exposed (Korean subjects). It is also known that EUV dose of greater than 360 J/m² can cause erythema. Therefore, for the purpose of the present invention the target UVB dose was set to below 360 J/m² assuming the skin exposed is 18%.

In view of the above, for the 4 modules tested, when radiant power of 13.4 mW, 39.6 mW, 72 mW, and 84 mW of UVB LED was applied for 9 hours, an EUVB energy dose of approximately 5.4 J/m², 53.46 J/m², 53.16 J/m², and 86 J/m² was provided, which clearly does not meet the target requirement.

However, unexpectedly and surprisingly, it was found that by including a reflector such that beam angle of 30° is achieved, radiant power of 39.6 mW and 84 mW UVB LED applied for 9 hours resulted in EUVB energy dose of 260 J/m² and 650 J/m² (measured at 1.8 m distance/height). Modules having radiant power of 13.4 mW and 72 mW were not selected for further evaluation as module having radiant power of 39.6 mW and 84 mW gave the most promising results as per requirements. While other beam angles were considered, they were not taken forward as the gain in EUVB dosage was not as promising as that obtained for 30° beam angle.

For the two modules of 39.6 mW and 84 mW UVB LED, after 9 hours exposure, the EUV dose was about 53.71 J/m² and 56.36 J/m² respectively at 1.8 m distance.

Table 2 shows the EUV dose at various distances for 84 mW (peak 310 nm).

| | Irradiance | | |
|---|---|---|---|
| Distance | (200-400 nm) | EUV | EUV Energy |
| (mm) | W/m\hat{ }2 | W/m\hat{ }2 | J/m\hat{ }2 |
| 200 | 4.92E−01 | 0.150570 | 4878.46 |
| 1000 | 2.23E−02 | 0.006778 | 219.61 |
| 1500 | 8.20E−03 | 0.002481 | 80.38 |
| 1800 | 5.69E−03 | 0.001740 | 56.36 |

Table 3 shows the EUV dose at various distances for 39.6 mW (303 nm).

| | UV | | |
|---|---|---|---|
| | Irradiance | | |
| Distance | (200-400 nm) | EUV | EUV Energy |
| (mm) | W/m\hat{ }2 | W/m\hat{ }2 | J/m\hat{ }2 |
| 200 | 1.63E−01 | 0.035714 | 1157.12 |
| 1000 | 1.41E−02 | 0.002069 | 67.03 |
| 1500 | 7.50E−03 | 0.001200 | 38.88 |
| 1800 | 6.15E−03 | 0.001658 | 53.71 |

Table 4 shows the actinic UV (AUV) hazard exposure for the skin and eye for 84 mW (peak 310 nm)

| | Irradiance | | |
|---|---|---|---|
| Distance | (200-400 nm) | AUV | |
| (mm) | (W/M\hat{ }2) | (W/M\hat{ }2) | Risk Group |
| 200 | 4.92E−01 | 0.0481 | Risk Group 3 |
| 1000 | 2.23E−02 | 0.00217 | Risk Group 1 |
| 1500 | 8.20E−03 | 0.0008 | Risk Group 0 |
| 1800 | 5.69E−03 | 0.00056 | Risk Group 0 |

Table 5 shows the actinic UV (AUV) hazard exposure for the skin and eye for 39.6 mW (peak 303 nm)

| | Irradiance | | |
|---|---|---|---|
| Distance | (200-400 nm) | AUV | |
| (mm) | (W/M\hat{ }2) | (W/M\hat{ }2) | Risk Group |
| 200 | 1.63E−01 | 0.015235046 | Risk Group 3 |
| 1000 | 1.41E−02 | 0.000786592 | Risk Group 1 |
| 1500 | 7.50E−03 | 0.00046986 | Risk Group 0 |
| 1800 | 6.15E−03 | 0.00043038 | Risk Group 0 |

Table 6 shows the near UV (NUV) hazard exposure for the skin and eye for 84 mW (peak 310 nm)

| | Irradiance | | |
|---|---|---|---|
| Distance | (200-400 nm) | NUV | |
| (mm) | (W/M\hat{ }2) | (W/M\hat{ }2) | Risk Group |
| 200 | 4.92E−01 | 0.08369 | Risk Group 0 |
| 1000 | 2.23E−02 | 0.0038 | Risk Group 0 |
| 1500 | 8.20E−03 | 0.0014 | Risk Group 0 |
| 1800 | 5.69E−03 | 0.00097 | Risk Group 0 |

Table 7 shows the near UV (NUV) hazard exposure for the skin and eye for 39.6 mW (peak 303 nm)

| | Irradiance | | |
|---|---|---|---|
| Distance | (200-400 nm) | NUV | |
| (mm) | (W/M\hat{ }2) | (W/M\hat{ }2) | Risk Group |
| 200 | 1.63E−01 | 0.0239 | Risk Group 0 |
| 1000 | 1.41E−02 | 0.0031 | Risk Group 0 |
| 1500 | 7.50E−03 | 0.001 | Risk Group 0 |
| 1800 | 6.15E−03 | 0.0007 | Risk Group 0 |

It can be observed that the measured values of AUV and NUV for 84 mW and 39.6 mW UVB LED at 1.8 m were 0.00056 W/m², 0.00043 W/m² and 0.00097 W/m², 0.00070 W/m² respectively, which are lower than the reference values of 0.001 W/² and 10 W/m².

For the two modules of 39.6 mW and 84 mW UVB LED (without reflector (100-110° beam angle), after 9 hours exposure, 96 IU and 101 IU of vitamin D can be generated respectively after exposure for 9 hours assuming that the EUV dose is about 53 J/m² and 56 J/m² respectively at 1.8 m distance and 18% exposed skin area for Indian skin type (type IV).

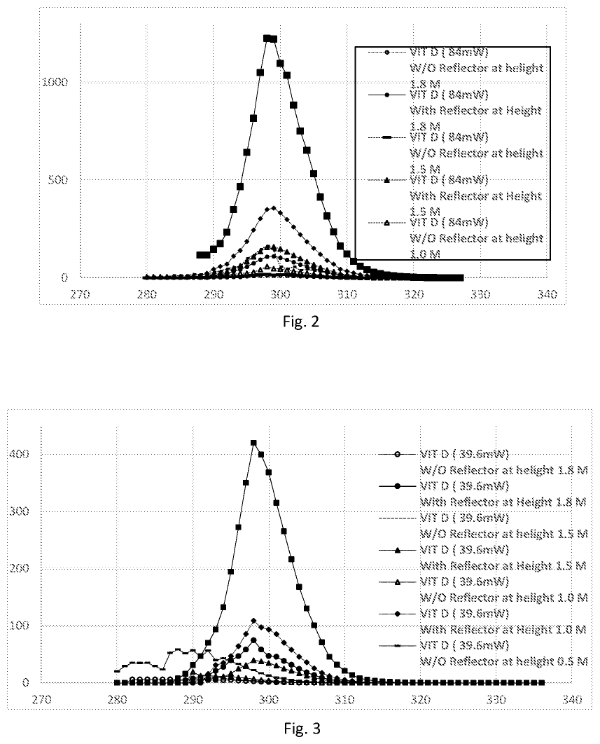

By addition of reflector to obtain a beam angle of 30°, the same amount of radiant power (84 mW), which is designed for peak wavelength of 310 nm produces 0.020033 W/m² of EUVB irradiance which produces 1168 IU of vitamin D during 9 hours of operation/exposure (778 IU after 6 hours exposure). However, this is beyond the safe levels and therefore generally not recommended for 9 hours exposure. FIG. 2 shows vitamin D levels after 9 hours from the said module with or without reflector as a function of distance.

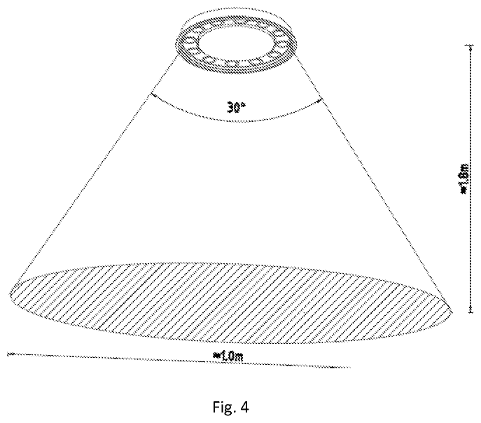

In the case of the 39.6 mW module, which is designed for peak wavelength of 303 nm, by addition of reflector to obtain a beam angle of 30°, 0.01004 W/m² of EUVB irradiance is achieved which produces 390 IU of vitamin D during 6 hours of operation/exposure (585 IU after 9 hours exposure). FIG. 3 shows vitamin D levels after 6 hours from the said module with or without reflector as a function of distance.

To achieve optimum lighting and vitamin D production, the lighting fixture of the present invention comprising UVB emitting LEDs having total radiant power of 39.6 mW and each LED having a peak wavelength of 303 nm, may be installed at a height of 1.8 m. The desired beam angle allows for sufficient UVB exposure covering a circular area of 1 m diameter (see FIG. 4). This also provides sufficient brightness of 500 lux in the designated coverage area.

In conclusion, the present invention provides a lighting fixture for application in indoor commercial spaces such as offices in particular, which provides safe exposure to UVB radiation for promoting vitamin D production by the human body. The UVB LEDs having peak wavelength of 303 nm and total radiant power of 39.6 mW meet all the safety standard and codes so as to be safe for general use and do not require any medical or professional supervision. The lighting fixture is designed based on generalized standardized settings such as distance of light source from person, exposed skin surface area, and time spent under the lighting fixture.

I/We claim:

1. A lighting fixture comprising:
a. one or more LEDs emitting light in the visible wavelength;
b. one or more LEDs emitting light in the UV spectrum having wavelength peak value of 303 nm; and
c. a reflector,
wherein upon exposure to UV light, AUV is not more than 0.00104 W/m², NUV is not more than 0.00056 W/m², and EUVB irradiance is not more than 0.01004 W/m² at 1.8 m from the light source.

2. The lighting fixture as claimed in claim 1, wherein the beam angle of one or more LEDs emitting light in the UV spectrum is 30°.

3. The fixture as claimed in claim 1, wherein the radiant power of a LED emitting light in the UV spectrum is 3.3 mW.

4. The fixture as claimed in claim 1, wherein the total radiant power of LEDs emitting light in the UV spectrum is 39.6 mW.

5. The fixture as claimed in claim 1, wherein the reflector is made of aluminum or PCTF.

6. The fixture as claimed in claim 1, wherein the visible light brightness of the fixture is in the range of 450-600 lux measured at 1.8 m from the light source.

7. A method of providing light in the UVB wavelength for promoting Vitamin D production on exposed skin, said method comprising:
a. obtaining at least a lighting fixture as claimed in claim 1;
b. exposing skin to said lighting fixture,
wherein said method promotes Vitamin D production.

8. The method as claimed in claim 7, wherein said method results in production of Vitamin D equivalent to 390 IU when skin (type IV) exposure is 18% of total skin and exposure time is 6 hours.

9. A lighting fixture comprising:
a. multiple LEDs emitting light in the visible wavelength;
b. 12 LEDs emitting light in the UVB spectrum having wavelength peak value of 303 nm; and
c. an aluminum reflector,
wherein the beam angle of LEDs emitting light in the UVB spectrum is 30°, the radiant power of a LED emitting light in the UV spectrum is 3.3 mW, and the visible light brightness of the fixture is 500 lux measured at 1.8 m from the light source.

10. The lighting fixture as claimed in claim 9, wherein upon exposure to UVB light, AUV is not more than 0.00104 W/m², NUV is not more than 0.00056 W/m², and EUVB irradiance is not more than 0.01004 W/m² at 1.8 m from the light source.
